(19) United States
(12) Patent Application Publication
(10) Pub. No.: US 2004/0018507 A1
(43) Pub. Date: Jan. 29, 2004

(54) Support plate and method for carrying out functional tests

(76) Inventors: Thomas Joos, Tubingen (DE); Dieter Stoll, Reutlingen (DE); Hansjurgen Volkmer, Tubingen (DE); Hugo Hammerle, Tubingen (DE); Manfred Schmolz, Gomaringen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/337,046

(22) Filed: Jan. 3, 2003

Related U.S. Application Data
(63) Continuation of application No. PCT/EP01/07720, filed on Jul. 5, 2001.

(30) Foreign Application Priority Data
Jul. 5, 2000 (DE) 10032730.3

(51) Int. Cl. C12Q 1/68; C12M 1/34

(52) U.S. Cl. 435/6; 435/287.2

(57) ABSTRACT

A support plate for carrying out functional tests on biological cells possesses raised sections which are spatially separated from each other and on which at least one measuring point, from an array of measuring points, is in each case arranged, with the cells being able to interact and bind at the measuring points. Trapping molecules, to which the cells are able to bind by means of their cell surface molecules, are immobilized at the measuring points, with the measuring points being separated from each other by regions of the support plate at which the cells cannot be immobilized (FIG. 4).

### RELATED APPLICATION

This is a continuation application of International Patent Application PCT/EP01/07720, filed Jul. 5, 2001, designating the United States and published in German as WO 02/02226 A2, which claims priority to German Application Number 100 32 730.3, filed Jul. 5, 2000.

### BACKGROUND OF THE INVENTION

1. Field of the Invention

The present invention relates to a support plate for carrying out functional tests on biological cells, having an array of measuring points at which the cells can interact and bind, with the measuring points being separated from each other by regions of the support plate at which the cells cannot be immobilized, and with trapping molecules, to which the cells can bind by means of their cell surface molecules, being immobilized at the measuring points.

2. Related Prior Art

A support plate of this kind is disclosed in WO 97/45730.

The known support plate contains a non-uniformly embossed array of chemicals on which cells are sown for the purpose of investigating the interaction of the chemicals with the cells. Further disclosed is a method for preparing such an array, in which method a support plate made of glass, plastic or silicone is treated chemically, thereby producing a surface which, taken overall, is hydrophobic and on which hydrophilic sites are then generated selectively using inkjet technology.

These hydrophilic sites, which are termed “wells”, contain functional groups which either themselves bind cells or are populated by molecules which for their part then bind cells.

It is possible to use such support plates for carrying out functional tests, as are known generally from the prior art, on biological cells; these methods are employed, for example, for pharmascreening.

The known methods are frequently carried out in what are termed microtitre plates which are small cell culture vessels possessing standardized, rectangular or round dimensions. The cell culture vessels possess wells which are arranged in rows and columns and into which the ligands, the cells and the test substances have to be pipetted individually. After incubation has taken place, the test result is analysed by analysing the microtitre plate either on its own or well by well. In this connection, it is possible for the measurement to be carried out, inter alia, either using a transmitted-light method or using a reflected-light method, with the optical density or the fluorescence of a reporter construct being taken, for example, as the measured parameter.

In this way, it is possible to measure the reaction of cells to particular pharmaceutical preparations, with it additionally being possible, in this connection, to stimulate the cells with particular ligands.

Particular disadvantages of the known method are that it is very time-consuming and that large quantities of substances are required.

On the other hand, an advantage of the known method is that there is no cross contamination between the individual wells since the individual test solutions are isolated from each other by the ridges between the individual wells.

The known method is not suitable, in particular, for carrying out a large number of measurements using cells which are derived, for example, from a biopsy and are therefore not available in any desired number.

In order to reduce the quantity of substance employed, U.S. Pat. No. 5,989,835 describes the use of what is termed a microplate which is 2033 30 mm in size and which has measuring points of from 100 to 200 μm in diameter and a centered distance of 500 μm. The microplates consist of coplanar layers of materials to which the cells to be investigated adhere. A pattern of other materials, to which the cells do not adhere, is affixed on these layers. This document does not describe the precise construction of these microplates; it is simply mentioned that the microplates can also exhibit a three-dimensional surface having a corresponding pattern and consequently essentially be microtitre plates possessing reduced dimensions.

As a result of the format being smaller than that of the microtitre plates, the quantity of substances employed is minimized and storage and handling during the experiments is facilitated. Furthermore, it is possible to achieve better optical resolution if the microplate is surveyed optically as a whole using a CCD camera.

According to this document, these microplates are employed in an automated method for analysing fluorescent-labelled cells whose reaction to different substances is to be tested, for example within the context of drug research.

For this, the adherent cells are treated with one or more substances and, after the incubation, are photographed at each measuring point using a fluorescence microscope. The optical data are then digitalized and an analysis is subsequently carried out to determine the effect which the substance has on the biological function being tested.

While the said document mentions the arrangement of measuring points on coplanar layers, microtitre plates of reduced format are employed in the implementation examples. While these microplates exhibit the abovementioned advantage with regard to economizing on substance and handling, the very small wells also involve disadvantages.

The inventors of the present application have found, on the one hand, that surface tensions increase as the wells become ever smaller, resulting in the cells under investigation being subjected to great stress. On the other hand, it is difficult to impossible to still introduce a homogeneous distribution of cells into small wells of from 100 to 200 μm in diameter, meaning, in contrast to what is stated in this document, that handling, particularly when pipetting, is made substantially more difficult.

The quantity of substance employed can also be reduced in the case of the support plate which is disclosed in document WO 97/45730 which was mentioned at the outset; however, the inventors of the present invention have found that the support plate which is described, and the method which can be carried out using it, suffer from the disadvantage that it is not possible to reliably prevent cross contamination between adjacent wells. Particularly when cell types which are in each case different are immobilized in the different wells in a small space in order, for example, to screen a test substance against different cell types, the disclosed test plate is disadvantageous since it is not possible to exclude cross contamination, which can lead to both falsely positive and falsely negative results.

### SUMMARY OF THE INVENTION

In view of the above, it is an object of the present invention to provide a support plate which is of the type mentioned at the outset and which avoids the abovementioned disadvantages. In particular, it should be possible to carry out reliable functional measurements on biological cells without having to employ large quantities of substances. In addition to this, it should also be possible to carry out a large number of different measurements and tests in parallel without increasing the amount of handling required or without there being any danger of cross contamination.

In the case of the support plate mentioned at the outset, this object is achieved, according to the invention, by the support plate possessing raised sections which are spatially separated from each other and on which at least one measuring point is in each case provided.

The object underlying the invention is in this way achieved in its entirety.

As a result of the raised sections, which can be designed in the manner of a peg which in each case possesses an essentially planar facing surface, cross contamination between measuring points arranged on different pegs is efficiently avoided. As a result of the spatial separation, that is as a result of recesses between the individual pegs or raised sections, cells which are insufficiently immobilized on a peg cannot reach measuring points on adjacent pegs; instead, they fall into the recesses between the pegs. Consequently, the novel construction of the support plate eliminates the danger, which exists in the case of planar support systems in the prior art, of cross contamination taking place between adjacent measuring points. This advantage can be augmented still further by selectively modifying the facing surface of the pegs with molecules which promote the adhesion of cells or particular cell types.

After the cells have been immobilized on the facing surfaces of the pegs, the support plate, together with the pegs, can be turned over. After non-adherent cells have been washed off, the support plate, with the pegs facing downwards, can be immersed in a vessel filled with a solution containing test substances in order to stimulate cells with test substances. It is correspondingly possible to coculture different cells, which are immobilized on the pegs, with other cells in the vessel. In this way, it is also possible to carry out a parallel screening of antibodies directly against cell surface molecules belonging to different cell, types, with a single cell type being immobilized on each peg. Known methods, which are described, for example, in WO 97/45730, can be used to detect changes in the cells immobilized on the pegs after the support plate, and consequently the pegs, have been removed from the vessel.

As a result of the arrangement of the support plate, with the pegs pointing downwards, which is selected when carrying out the biological tests which are described above by way of example, the possibility of cross contamination is reduced still further since non-immobilized/adherent cells fall off the support plate as a result of the effect of gravity such that it is only immobilized cells, which have been costimulated, on the one hand, for example, by trapping molecules (ligands, antibodies, etc.) which are immobilized on the pegs and, on the other hand, by test substances present in the vessel, which are still present on the measuring points after the incubation with the test solution and the removal of the support plate.

It may also be mentioned that, in the simplest case, the trapping molecules constitute a surface coating on which cells can grow in an adherent manner.

However, as a result of immobilizing the biological cells to be investigated by way of trapping molecules which are for their part immobilized on the facing surfaces of the pegs, it is possible to use the novel support plate not only for investigating adherent cells but also for investigating non-adherent cells, such as T cells, which can be immobilized by way of their cell surface receptors using specific trapping molecules.

The problem of applying small quantities of cell suspension to the facing surfaces of the pegs can be solved by using micrometering systems to coat these facing surfaces with trapping molecules to which only particular cells, belonging to one or more cell types, from the cell suspension bind. Handling is markedly facilitated by the fact that the cells no longer have to be applied successively to the measuring points but merely have to be loaded onto the support plate, which can be designed, for example, as a cell culture vessel baseplate carrying pegs or stamps. In this way, only a single pipetting step is required for supplying all the measuring points of an array with cells.

The pegs, and consequently the measuring points, are now located in a large well such that the problem of surface tension, as known from the prior art, is eliminated, with, on the other hand, it also being readily possible to achieve a homogeneous distribution of the cells in this large well without the cells having to be exposed to mechanical stress.

In order to create an alternative possibility for rapidly and simply coating the facing surfaces of the pegs with different trapping molecules, and for applying different cells, a further object of the invention relates to a metering plate for a novel support plate, with the metering plate possessing drilled holes which correspond to the pegs such that, when the metering plate has been mounted on, the pegs seal off, by means of their facing surfaces, the drilled holes towards the bottom and form cavities/wells in the drilled holes.

Before the cells are applied, a well which is specific to it is, as it were, formed over each peg, which well is used for coating and/or applying cells on the facing surface of the pegs. In this connection, the pegs can be of conical design, at least in the region of their facing surface, with the drilled holes in the metering plate also being conical in a corresponding manner such that it is possible to achieve good sealing at the bottom of the wells which are formed when the metering plate is mounted on the pegs.

In other words, the facing surface of the pegs serves as the bottom of a well in a metering plate which possesses drilled holes in the grid pattern of the pegs and which has been mounted on the support plate. The wells can be filled with trapping molecules and cells using established metering systems. After the metering plate has been removed, the pegs, which have been coated and populated with cells, serve once again as raised structures as has been described above. In this way, it is possible for different pegs, i.e. different measuring points, to be simply, rapidly and reliably coated with different trapping molecules and populated with different cells.

By these means, identical or different trapping molecules, and identical or different test substances, can consequently be immobilized at the measuring points.

In this way, it is possible, for example, to prepare an array of measuring points which can be used to carry out a very wide variety of measurements simultaneously on identical cells. This also covers costimulation by the trapping molecules, which comprise the ligands, and the test substances.

According to another object, the test substances are immobilized at the measuring points and it is not absolutely necessary to arrange the measuring points on pegs; in this case, it is also possible to separate the regions carrying the different measuring points in the manner disclosed in the initially mentioned WO 97/45730.

In the light of this document, the immobilization of test substances at the measuring points is also not only novel but inventive in itself since the costimulation of the cells by test substances and trapping molecules/ligands can be examined in a simple manner. In this connection, the danger of cross contamination can be substantially reduced by suitable selection of the experiment.

It is furthermore also possible to add the test substances in solution and consequently not immobilize them at the measuring points.

According to this object, the test substance can be a pharmaceutical active compound whose influence on the immobilized cells is being tested or an antibody which is being screened against cell surface receptors on the cells. When antibodies are being screened, different cell types are in each case immobilized on the different pegs such that it is possible, by means of a site-resolved measurement, to determine which cell types possess surface receptors for the antibody being investigated.

According to another object, it is also possible for the test substance to comprise cells whose interaction with the immobilized cells is to be examined. In principle, this measurement method is disclosed, as coculturing, in the prior art.

In the known coculturing methods, different cell types from the same or different tissues are cultured in a common nutrient solution, with the different cells being arranged so that they are separated from each other by means of a membrane or a diffusion distance filled with medium in order to prevent direct contact between the cell membranes of the different cell types since this has a negative influence on the measurement result. A disadvantage of the known methods and devices for carrying out coculture measurements of this nature is that they are very labour-intensive and cost-intensive such that the measurements can only be carried out in parallel in small numbers.

When the novel support plate is used, it is also possible, due to the fact that it is readily possible to miniaturize the peg dimensions, to adapt coculturing methods to test formats, i.e. plates containing 96 or 384 wells, which are capable of high throughput. In addition, the support plate can also be used to carry out novel coculturing methods in which the pegs are coated, for example, with one cell type or several cell types. These cells can, of their own accord, be subject to a partial inhibition, as a result of being pretreated, such that it is only a cell monolayer which is formed on the facing surface of the pegs. This avoids cross contamination between the cells on adjacent pegs and contamination of the cell culture which is present in the vessel due to cells falling down from the pegs. In this way, the cells are spatially separated from each other over the shortest possible distance, thereby making it possible to carry out informative measurements.

Different cell types can be immobilized on adjacent pegs, with a further cell type being present in the culture vessel into which the pegs are then immersed from above. In this way, it is also possible to carry out multivariant cocultures which were not previously possible to implement and in which it is possible to examine the interaction on each other of more than two cell types in a simple test vessel using different cell types which are spatially separated in the same culture vessel. For example, the system which has been described can be used to carry out coculturing experiments which employ spatially separated reference cells and test cells which are immobilized on adjacent pegs in the same cell culture vessel and in which the reference cells constitute an internal standard which, in the biological test, was exposed to conditions which were identical to those to which the cells to be analysed were exposed. The fact that the test results from the analysed cells are directly related to results obtained with the reference cells following culturing in the same environment facilitates the interpretation of the measured data and reduces the spread of the test results.

In addition to this, dose-effect curves of, for example, pharmacologically or toxicologically active substances, or other biologically active compounds, can be tested if, for example, these substances are additionally added to the solution, with the substances being able to affect the interaction between the immobilized cells and the cells which are present in solution.

In general, it is possible, for example, to bind different cells from a non-homogeneous cell suspension to individual measuring points by way of cell-specific trapping molecules. It is possible, for example, to measure axonal growth in the presence and absence of integrin-specific antibodies or to determine cytokine secretion by T cells.

In the context of an allergy test, it is also possible to immobilize particular antigens on the measuring points and to use whole blood or cell fractions as the cell suspension in order to check whether the individual from which the whole blood is derived reacts to the allergens.

If the reaction of a particular cell line to a particular test substance is to be examined, different trapping molecules are immobilized at the measuring points/pegs in order to be able to test the reaction to the test substance when the interaction with the environment varies.

It is naturally necessary for the arrangement of the different trapping molecules and/or test substances in the array to be known. To ensure this, the array is prepared using conventional appliances for contact printing or inkjet technology which enable measuring points having very small diameters and very small edge spacings to be generated.

In this connection, U.S. Pat. No. 5,985,551 describes the preparation of an array of functionalized binding sites on a support surface. In this connection, an array from 10 to 10⁴ sites/square centimetre is defined on a covalently bound layer of inert siloxane, with this covalently bound layer not being present at these sites. The sites have a diameter of 50-2000 μm, with chemical reaction solutions being localized at these measurement sites as a result of surface tensions.

The applications which are described are oligonucleotide probe arrays and peptide arrays, which can be used to carry out a variety of analyses. In this case, cross contamination is prevented by each measurement site being surrounded by its own hydrophobic wall.

In the case of the novel support plate, preference is given, in this connection, to the measuring points having a diameter of between about 200 μm and about 4000 μm, with the measuring points preferably having an edge spacing of between about 300 μm and about 700 μm.

Preference is furthermore given to each peg having an area of 1-5 mm² at its facing surface.

In this way, it is possible to generate very compact microarrays having very small measuring points which are, however, arranged on essentially planar, raised sections of the support plate such that they do not form any wells but, instead, lie in a large well.

While this approach minimizes the use of the substances, on the one hand, it on the other hand avoids the disadvantages which are associated with small wells and which relate to handling when pipetting and to surface tensions and stress for the cells.

In this connection, any assay which provides information about cell properties in a site-resolved manner can be used for the readout. For example, GFP reporter assays, morphometric analyses (axonal growth, differentiation, proliferation), cytoskeletal changes, movements, etc., can be observed.

Furthermore, it is possible to implement function tests for cell culture cells following gene transfer in order to investigate which gene product alters the morphology. In addition to this, it is possible to carry out an adhesion and proliferation screening, for example for characterizing cancer cells. Furthermore, the potential for metastasis can be assessed and the differentiation state can be determined. In addition to diagnosis and cancer cell screening, it is also possible to test substances or examine the influence exerted by second messenger systems by way of ECM receptors.

Taken overall, the novel support plate offers an unlimited possibility for carrying out functional investigations on biological cells, in connection with which the quantity of substance used is small and handling is simple.

In this connection, preference is given to selecting the trapping molecules from the group: proteins, such as components of extracellular matrix proteins (fibronectin, laminin, collagen, cell surface proteins, receptors and ligands), polylysine, peptides from laminin sequences, control peptides, control proteins, peptidomimetics, polymers, lectins, antibodies, antigens and allergens.

The test substances are preferably selected from the group: pharmaceutical preparations, antibodies, substances which exert an influence on cell properties, messengers, growth factors and antigens.

Particular preference is given to the support plate being made from a material which is selected from the group: glass and plastic, in particular polystyrene and/or silicone.

When this is done, it is advantageous that transmitted light measurements can also be readily carried out, with it being possible, in a preferred manner, to provide functionalized surfaces, on which the trapping molecules and/or test substances can easily be immobilized, on glass and plastic. It is furthermore readily possible to block the functionalized surfaces in the regions between the pegs and/or measuring points such that no cells are able to adhere to these regions.

In this connection, the functionalized surface is selected from the group: aldehyde-activated surface, epoxide-activated surface, aminosilanized surface, poly-D-lysine-coated surface and protein-coated polystyrene surface.

Preference is furthermore given to cells which have not been immobilized being removed, by washing the support plate, before the cell properties are detected.

In this way, too, it is possible to even more effectively prevent cells as it were migrating from one measuring point to another and in this way simulating a false result.

The novel support plates can be prepared for particular assays and marketed commercially as such. In this connection, it is possible to prepare support plates for allergy tests, cancer cell screening, etc., by providing the support plates with particular trapping molecules, in connection with which it is furthermore possible to also immobilize test substances on the measuring points/pegs together with the trapping molecules. However, it is also possible to supply the test substances together with the support plates in the manner of a kit.

In this connection, preference is given to the support plates preferably being sterilized by being irradiated with UV light or gamma radiation, with the plates furthermore preferably being rehydrated by being incubated with PBS and in addition preferably being packed moist.

In this way, it is possible to store a prepared support plate over weeks and months without it being contaminated or without there being any risk of it drying out.

When using the novel support plate, it is consequently possible, for the first time, to carry out standardized assays and to acquire the support plates used for these assays, and possibly also the test substances in solution, as a kit.

In the light of the above observations, the present invention furthermore relates to a method for carrying out functional tests on biological cells in which the cells are applied to a novel support plate, the immobilized cells are cultured in a solution which contains a test substance whose influence on the cells is to be investigated and the cells which have been influenced are detected in a site-resolved manner.

In one embodiment in this connection, those cell properties which can be influenced by costimulating the cells with trapping molecules and test substances are detected in a site-resolved manner.

In another embodiment, the test substance can comprise antibodies which are screened against surface receptors on the cells.

In another embodiment, the test substance can comprise cells whose interaction with the immobilized cells is being investigated, with it being possible for the solution to additionally contain substances which influence the interaction.

Other advantages ensue from the description and the enclosed drawing.

It will be understood that the features which are mentioned above, and those which are still to be explained below, can be used not only in the combinations which are in each case indicated but also in other combinations, or on their own, without departing from the scope of the present invention.

### BRIEF DESCRIPTION OF THE DRAWINGS

Embodiments of the invention are depicted in the drawing and are explained in more detail in the description which follows.

FIG. 1 shows a diagrammatic view from above of a support plate with measuring points;

FIG. 2 shows a section, which is not to scale, through the support plate shown in FIG. 1;

FIG. 3 shows an embodiment of a novel support plate depicted as in FIG. 2;

FIG. 4 shows a support plate according to the invention, together with pegs, which is designed as the bottom plate of a cell culture vessel and which is depicted as in FIG. 2;

FIG. 5 shows the use of the support plate from FIG. 4, depicted as in FIG. 3;

FIG. 6 shows a support plate as in FIG. 4, which support plate is provided with a removable metering plate; and

FIG. 7 shows a perspective representation of the support plate and metering plate from FIG. 6 in the assembled state and the disassembled state.

### DESCRIPTION OF PREFERRED EMBODIMENTS

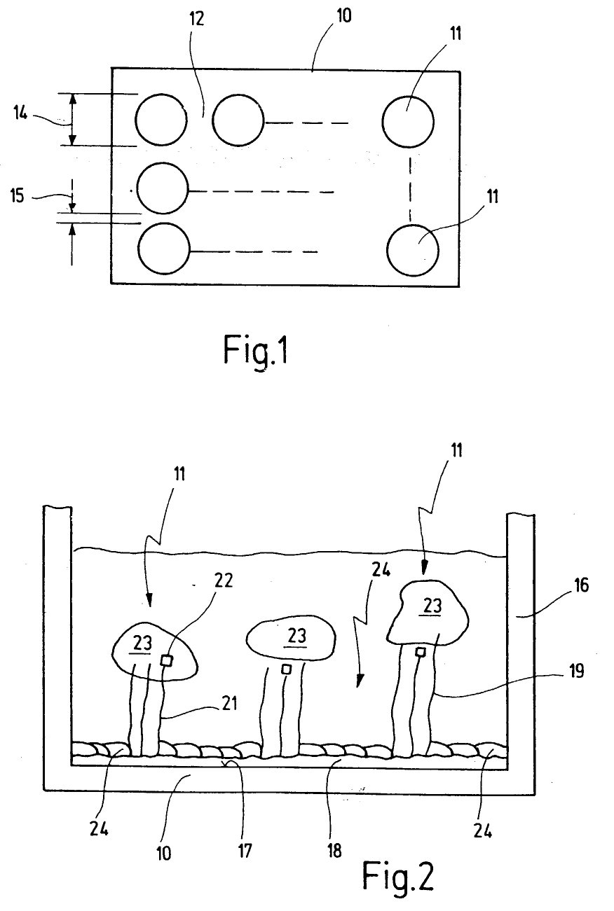

In FIG. 1, 10 denotes a rectangular support plate which is made of glass or plastic and on which, by way of example in this present case, some measuring points 11 are arranged, at which biological cells, which are not shown in FIG. 1, can be immobilized. Regions 12 of the support plate 10, on which regions the biological cells do not immobilize, are provided between the measuring points 11.

The measuring points 11 have a diameter 14 of from 600 to 800 μm and an edge spacing 15 of from 400 to 200 μm, such that a center distance of 500 μm is obtained. In this way, 100 measuring points can be accommodated on a support plate 10 having an edge length of 1 cm.

As is shown in the diagrammatic side view depicted in FIG. 2, the support plate 10 can be designed as the bottom plate of a cell culture vessel 16. On its planar section 17, the support plate 10 carries a functionalized surface 18 at which trapping molecules 19 are immobilized in the measuring points. In addition, test substances 22 are immobilized in the measuring points by way of immobilized linkers 21.

Biological cells 23, whose reaction to costimulation by the trapping molecules 19 and the test substances 22 is to be investigated, bind to the trapping molecules 19 and to the test substances 22.

In the regions 12 between the measuring points 11, the functionalized surface 18 is blocked with molecules 24 such that the cells 23 can only be immobilized in the region of the measuring points 11.

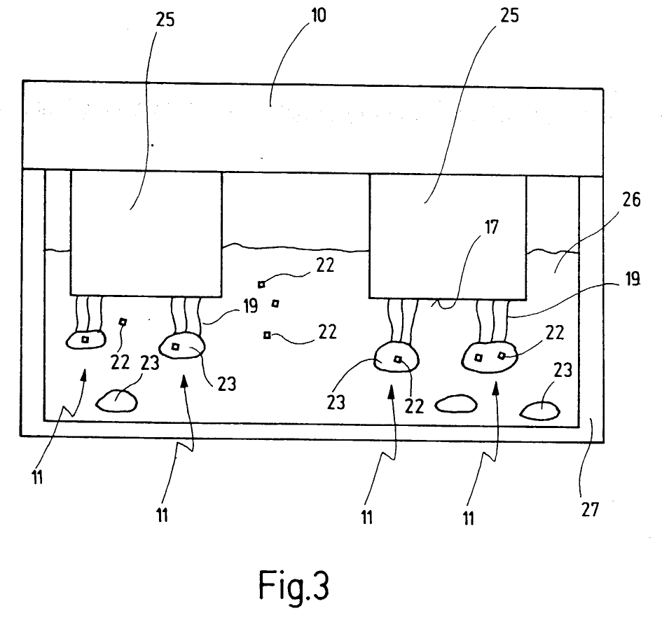

FIG. 3 shows an embodiment according to the invention of the support plate 10 which, in this present case, carries raised regions in the form of pegs 25 on each of which a planar section 17 is provided as the facing surface, which planar section 17 is provided, as shown in FIG. 2, with a functionalized surface on which the individual measuring points 11 are formed. While the area of the section 17 is, in this present case, 1.6×1.6 mm², for example, it can also be markedly smaller. For example, the height of a peg can be 1 mm.

In the example shown, the measuring points 11 only contain trapping molecules 19, to which cells 23 become attached; the test substances 22 are present in a solution 26 which is contained in a culture vessel 27.

The support plate 10, together with the planar sections 17, has been laid downwards from above on the culture vessel 27 such that the pegs 25 project into the solution 26. As the result of gravity, cells 23 which are not immobilized now become detached from the pegs 25 whereas the cells 23 which are immobilized at the measuring points 11 interact with the test substances 22 which are present free in the solution 26.

After the incubation has come to an end, the support plate 10 is removed upwards from the culture vessel 27, with only those cells 23 which have undergone costimulation by the trapping molecules 19 and test substances 22 remaining on the pegs 25. This avoids any cross contamination between adjacent measuring points 11.

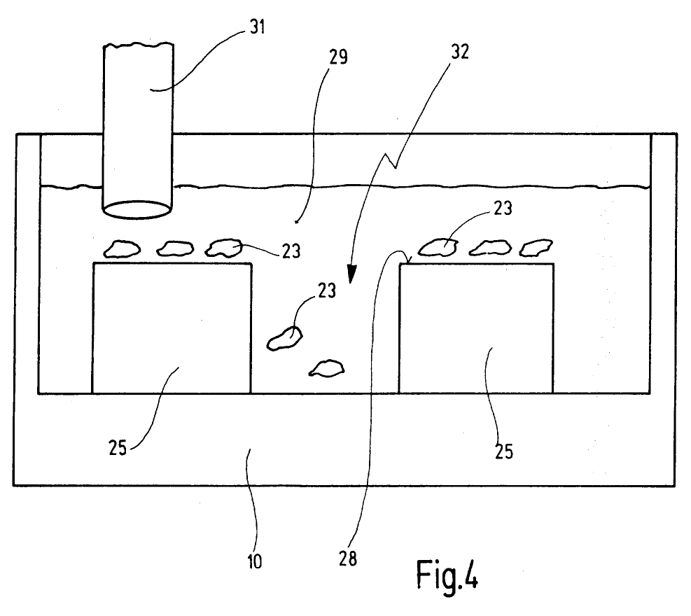

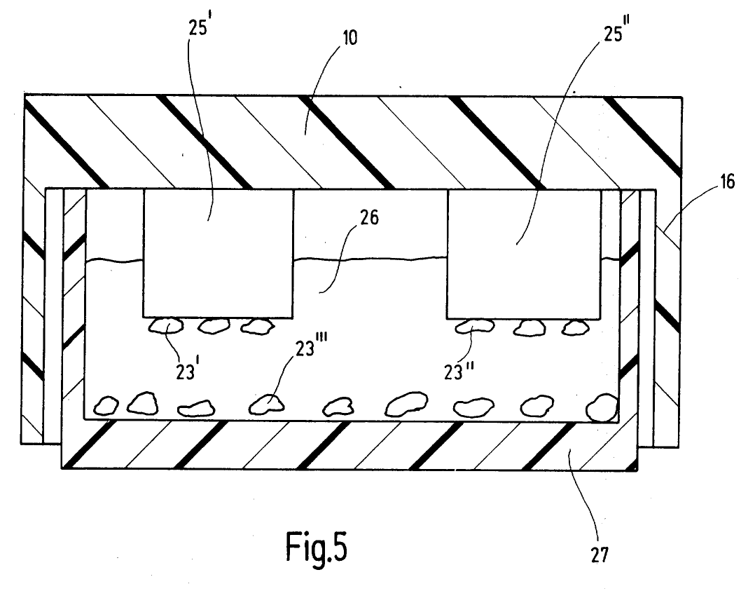

FIG. 4 shows a support plate 10 which is depicted as in FIG. 2 and which is designed as the bottom plate of a cell culture vessel 16. In FIG. 4, pegs 25, which possess a functionalized surface, to which cells 23 adhere, on the facing surface 28, are arranged in accordance with the invention on the support plate 10. In FIG. 4, the pegs 25 are completely immersed in a solution 29. The cells 23 can be applied, on the one hand, by the cells 23 being present free in the solution 29 and settling uniformly on the facing surfaces 28. In this case, all the pegs 25 are colonized with cells of an identical type.

On the other hand, the facing surfaces 28 of different pegs 25 can always be colonized with cells of differing type, to achieve which the cells are individually added to the facing surfaces 28, where they adhere, using a capillary which is only indicated diagrammatically at 31. Cells 23 which do not adhere and/or which are added in excess sink into recessed interstices 32 which spatially separate the pegs 25 from each other.

This thereby ensures that the desired cells 23 are immobilized on the facing surfaces 28.

It should also be pointed out, solely for the sake of completeness, that the measuring points are separated from each other on the facing surface 28 of a peg 25 by the functionalized surface 18 shown in FIG. 2 being blocked in the regions 12 between the measuring points 11. The interstices 32 between the pegs 25 give rise to a further, spatial separation which prevents cells 23 from one region of measuring points 11 reaching another region of measuring points 11, something which could lead to cross contamination and consequently to falsification of the measurement results.

Test substances, for example, whose reaction on the cells 23 is to be tested, can now be added to the solution 29. After an appropriate incubation, the solution 29 is removed, after which particular properties of the cells 23 on the pegs 25 are measured in a site-resolved manner, for which purpose it is also possible, for example, to use reporter assays.

If different cell types are immobilized on the pegs 25, the differentiation, proliferation or apoptosis of the cells, or the ability of the cells to specifically bind antibodies which are present in the solution 29 to surface antigens, can be measured in parallel in the same cell culture vessel 27. As a reference, isolated surface antigens can be immobilized on another peg in the same culture vessel 27. A third peg can furthermore be used for determining the total antibody concentration in the solution 29. To do this, it is possible, for example, to immobilize second antibodies, which are directed against constant regions of the tested first antibodies in solution, on this peg. In this way, it is possible to carry out a rapid and parallel screening of antibody specificities for different native cell surface molecules.

In addition to this, it is possible to add cells of another type, which cells interact specifically with the immobilized cells, to the solution 29, with it being possible to exert further influence on the interaction by means of added test substances.

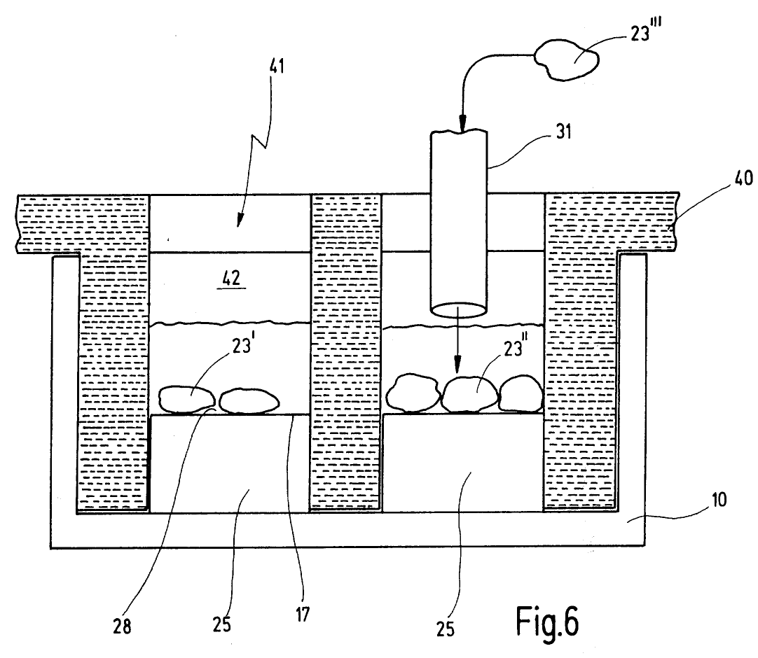

In order to ensure that no physical contact is established between the immobilized cells and the other cells which are present in solution 29, the cell culture vessel 16 from FIG. 4 can, in a similar way to that shown in FIG. 3, be inserted upside down, as shown in FIG. 5.

Cells 23′ are immobilized on the peg 25′ while cells 23″, of another type, are immobilized on the peg 25″.

Cells 23′″ of a third type, which cells, for example, are producing particular factors which act on the cells 23′ and 23″, are located at the bottom of the cell culture vessel 27.

In this way, it is possible to analyse cocultures, in which cells of different tissue types influence each other by releasing particular factors. Examples of cocultures which can be analysed using the setup shown in FIG. 5 are cellular models for test systems for the blood/brain barrier in which the ability of active compounds to penetrate the barrier is investigated and endothelial cells and astrocytes are employed.

Namely, the endothelial cells require factors, which are formed by the astrocytes, in order to develop specific blood/brain barrier functions. Particular test substances can influence the interaction between astrocytes and the endothelial cells, and this influence can be investigated in a site-resolved manner.

These cocultures can be carried out without a diffusion barrier (membrane), in which case it is possible, for example, to produce the support plates 10 by the injection moulding technique. It is particularly advantageous that it is possible to coculture several different immobilized cell types, rapidly and simply and in the same cell culture vessel, with a cell type which is present in solution, such that it is possible to carry out a multiparametric analysis under culture conditions which are absolutely identical for all the cells. This furthermore makes it possible to establish a reference to an internal reference cell line.

After the assay has been carried out, the support plate 10 is removed from the cell culture vessel 27 and specific parameters of the cells 23′ and 23″, which are immobilized on the pegs 25′, 25″, are measured in a site-resolved manner. If cells 23′, 23″ should become detached from the pegs 25′, 25″ during the measurement, they then remain in the cell culture vessel 27 and are not codetected. In this way, the only cells 23′, 23″ to be measured are those which have also actually participated in the assay.

FIG. 6 depicts a support plate 10, as in FIG. 4, on which a metering plate 40 has been mounted. The metering plate 40 possesses drilled holes 41 in the same grid pattern as the arrangement of the pegs 25, with the drilled holes 41 having a greater depth than that corresponding to the height of the pegs 25. When the metering plate 40 is mounted on the support plate 10, the drilled holes 41 are pushed, in a flush-fitting manner, over the pegs 25, which can have a slightly conical shape, such that they taper upwards, for this purpose. Naturally, the drilled holes 41 also have to have a corresponding conical shape.

In this way, the facing surfaces 28 of the pegs 25 become the bottoms of wells 42, which can be filled with liquids or cell suspensions, of one cell type or of different cell types, using a pipette or a capillary 31. After having finished the cell culture in the wells, that is after the cells have been immobilized on the facing surfaces 28, the metering plate 40 is removed from the support plate 10. The pegs 25 can then be used in the manner which was described above in connection with FIGS. 3 and 5. Using the metering plate 40 makes it possible to carry out simple selective modifications of individual pegs 25 without adjacent pegs 25 being contaminated. In this connection, the system can be adapted to existing methods for automated liquid handling. At the same time, the method offers the advantages, which have already been described, of a simple, rapid and parallel assay in macrovessels.

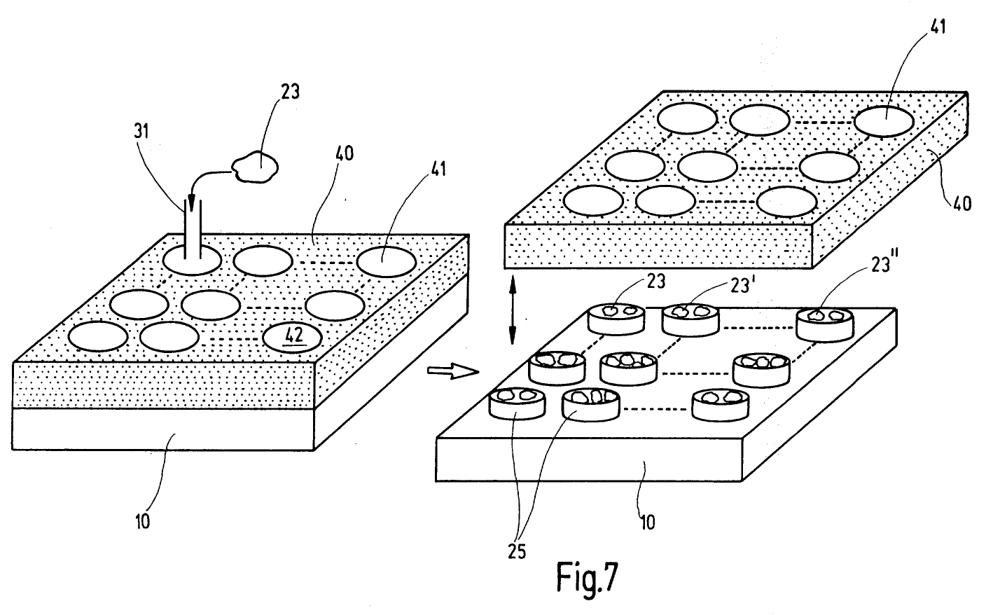

FIG. 7 shows, on the one hand, a perspective representation of the state in which the metering plate 40 has been mounted on a support plate 10 and, on the other hand, again as a perspective representation, the state in which the meeting plate 40 has been taken off the support plate 10 once again and cells 23′, 23″ are immobilized on the pegs 25.

### EXAMPLE 1

Producing Support Plates Possessing Measuring Points

Glass supports, polystyrene supports or silicone supports which are aldehyde-activated or aminopropylsilane-modified or polylysine-modified, and which possess pegs on which peptides are immobilized as trapping molecules, are used as support plates.

The peptides are prepared, in accordance with established methods (Fmoc strategy), on a solid phase, with, in each synthesis cycle, the N termini of potentially unextended peptide sequences being blocked, by a reaction with acetic anhydride, for further couplings with amino acid building blocks prior to the temporary Fmoc protecting group being cleaved off. At the end of the synthesis, a group by way of which the peptide can be bound specifically to surfaces (e.g. thiol function in cysteine) is incorporated at the N terminus of the complete peptide sequence. Since this ensures that incomplete peptide sequences can no longer be bound to the surfaces, the crude peptides are purified in connection with the surface modification. This makes it possible to avoid any elaborate preliminary HPLC purification.

The array of measuring points can be prepared using two methods:

1) Before being applied, peptides are bound covalently to carrier proteins (BSA, polylysine) and spotted, together with the other components which are required, onto aldehyde-activated pegs or plastic surfaces (from 10 to 30 nl/measuring point, 0.1-35 mg/ml). The peptides are immobilized by forming Schiff's bases, which are reduced to amine using NaCNBH₃. Binding to plastic surfaces is achieved by way of hydrophobic interactions.

2) Polylysine solution (10-30 nl, 1 mg/ml) is spotted onto aldehyde-activated or hydrophobic pegs/surfaces. The amino groups of the polylysine which has been applied are then reacted with SMPB, for which purpose the complete support is incubated with 5 mM sulfo-SMPB (2.3 mg/ml in distilled water) at room temperature for 2 hours and subsequently washed twice for 5 minutes with distilled water. Since the SMPB only reacts with amino groups of the polylysine, the remaining aldehyde groups on the support are not caused to react in this connection. Components of extracellular matrix proteins can, for example, be bound covalently, as test substances, to the glass support by way of these aldehyde groups in a second step.

The spotting is carried out using conventional appliances for inkjet technology or contact printing.

For the purpose of coupling the peptides to the SMPB-activated polylysine spots, the support is adjusted in the appliance such that the peptides to be spotted can be positioned precisely on the SMPB-activated polylysine spots. For this, the peptides are dissolved in 10% DMF/90% PBS (dissolved in DMF).

ECM components, laminin, fibronectin, collagen or polylysine (10-30 ml, 1-25 mg/ml) are spotted onto regions of the measuring points which are not occupied by polylysine.

Peptides or proteins which are not covalently bound are removed by washing the support and free aldehyde groups are blocked with serine (1 mg/ml) or H₂N-PEG (2 mg/ml) in NaCNBH₃ (0.1 M). These groups prevent/minimize any adhesion of the biological cells outside the measuring points.

For sterilization, the support, together with the array, is irradiated with UV light for 5 minutes under a laminar air flow hood. The array is subsequently rehydrated with PBS in a Petri dish for 10 minutes and then packed moist.

### EXAMPLE 2

Measurements Performed on Chick Tectum Cells

A cell suspension of chick tectum cells is applied, in 0.5 ml of solution, to the array and test substances which interfere with axonal growth are added in parallel or subsequently. After 24 hours, cells which are not bound are washed away.

The bound cells are assessed with regard to their specific adherence to the measuring points and with regard to vitality and specific differentiation features (polarity/axonal growth).

For this, the cells are fixed and labelled with an axon-specific antibody (anti-neurofilament Ab, anti-Tau Ab). This makes it possible to read out and quantify axonal growth automatically.

Using this assay, it is possible to test axonal growth in the presence and absence of integrin-specific antibodies. In this connection, it was possible to show that, while the antibody W1B10 (anti-β1 integrin) prevents β1-dependent axonal growth on laminin and fibronectin, N-CAM- and N-cadherin-dependent axonal growth is not affected.

### EXAMPLE 3

T cell Activation

The purpose of this experiment is to measure the T cell response following antigen stimulation. For this, the antigen is immobilized on the support plate in the manner which has already been described, with a signal amplification being achieved by immobilizing the antigen together with the T cell-stimulating surface receptor antibody (anti-CD28 costimulator). It is known that, on its own, anti-CD28 does not stimulate T cells.

The measurement parameter employed is that of determining cytokines after the T cells have been activated. This determination can be carried out by using standard Elisa tests to detect secreted cytokines or by measuring the cytokine which has accumulated intracellularly after release has been blockaded (brefeldin A).

If cytokine release, but not cytokine synthesis, is stopped by adding secretion-blocking agents, the cytokine which is synthesized, but not secreted, by the T cell can be detected in the cell. If different antigens are immobilized in the measuring points, the T cell response to the different antigens can be observed in a site-resolved manner.

What is claimed is:

1. A support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules, to which the cells can bind by means of the cell surface molecules thereby being immobilized at the measuring points, and
raised sections which are spatially separated from each other and on which at least one measuring point is in each case provided.

2. The support plate according to claim 1, wherein said trapping molecules comprise ligands for cell surface receptors.

3. The support plate according to claim 1, wherein identical trapping molecules are immobilized at the measuring points.

4. The support plate according to claim 1, wherein different trapping molecules are immobilized at the measuring points.

5. The support plate according to claim 1, wherein a test substance is immobilized at the measuring points.

6. The support plate according to claim 5, wherein identical test substances are immobilized at the measuring points.

7. The support plate according to claim 5, wherein different test substances are immobilized at the measuring points.

8. The support plate according to claim 1, wherein the plate is sterilized.

9. The support plate according to claim 8, wherein the plate is sterilized by being treated with radiation selected from the group consisting of UV light and gamma rays.

10. The support plate according to claim 1, wherein the plate is rehydrated.

11. The support plate according to claim 10, wherein the plate is rehydrated by being incubated with Phosphate-Buffered Saline.

12. The support plate according to claim 1, wherein the plate is packed moist.

13. The support plate according to claim 10, wherein the plate is packed moist.

14. The support plate according to claim 11, wherein the plate is packed moist.

15. The support plate according to claim 1, wherein each raised section is designed in the manner of a peg and is essentially planar at its facing surface.

16. The support plate according to claim 1, wherein the measuring points have a diameter of between about 200 μm and about 4000 μm.

17. The support plate according to claim 1, wherein the measuring points have an edge spacing of between about 300 μm and about 700 μm.

18. The support plate according to claim 16, wherein the measuring points have an edge spacing of between about 300 μm and about 700 μm.

19. The support plate according to claim 15, wherein each peg has an area of 1-5 mm² at its facing surface.

20. The support plate according to claim 18, wherein each peg has an area of 1-5 mm² at its facing surface.

21. The support plate according to claim 1, wherein the trapping molecules are selected from the group consisting of proteins, such as components of extracellular matrix proteins (fibronectin, laminin, collagen, cell surface proteins, receptors and ligands), polylysine, peptides from laminin sequences, control peptides, control proteins, peptidomimetics, polymers, lectins, antibodies, antigens and allergens.

22. The support plate according to claim 5, wherein the test substances are selected from the group consisting of pharmaceutical preparations, antibodies, substances which exert an influence on cell properties, messengers, growth factors, antigens and allergens.

23. The support plate according to claim 1, wherein the plate is made from a material selected from the group consisting of glass and plastic.

24. The support plate according to claim 23, wherein said plastic is selected from the group consisting of polystyrene and silicone.

25. The support plate according to claim 1, wherein the plate has a functionalized surface at least on the raised sections.

26. The support plate according to claim 25, wherein the functionalized surface is selected from the group consisting of aldehyde-activated surface, epoxide-activated surface, aminosilanized surface, poly-D-lysine-coated surface and protein-coated polystyrene surface.

27. The support plate according to claim 26, wherein the functionalized surface is blocked in the regions between the measuring points.

28. The support plate according to claim 1, wherein the plate is designed as a peg-carrying bottom plate of a cell culture vessel.

29. A support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules to which the cells can bind by means of the cell surface molecules thereby being immobilized at the measuring points, and
raised sections which are spatially separated from each other and on which at least one measuring point is in each case provided,
wherein the trapping molecules comprise ligands for cell surface receptors, and the measuring points have a diameter of between about 200 μm and about 4000 μm, and an edge spacing of between about 300 μm and about 700 μm.

30. A support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules to which the cells can bind by means of the cell surface molecules thereby being immobilized at the measuring points, and
raised sections which are spatially separated from each other and on which at least one measuring point is in each case provided, wherein each raised section is designed in the manner of a peg and is essentially planar at its facing surface, and
wherein the trapping molecules comprise ligands for cell surface receptors, and the measuring points have a diameter of between about 200 μm and about 4000 μm, and each peg has an area of 1-5 mm² at its facing surface.

31. A method for carrying out functional tests on biological cells, comprising:
applying the cells to a support plate of claim 15,
culturing the cells, which are immobilized on the raised sections of said support plate, in a solution which comprises a test substance whose influence on the cells is to be investigated, and
detecting, in a site-resolved manner, the cells which have been influenced by said test substance.

32. The method according to claim 31, wherein the cell properties which can be influenced by culturing the cells in the presence of the trapping molecules and the test substance are detected in a site-resolved manner.

33. The method according to claim 31, wherein the test substance comprises antibodies which are screened against surface receptors on the cells.

34. The method according to claim 31, wherein the test substance comprises cells whose interaction with immobilized cells is being investigated.

35. The method according to claim 34, wherein the solution furthermore comprises substances which influence said interaction.

36. The method according to claim 31, wherein, after the incubation, the support plate is dipped, facing downwards, from above into a vessel containing a solution comprising the test substance in order to exert an influence on the cells.

37. The method according to claim 31, wherein cells which have not been immobilized are removed, by washing the support plate, before the cell properties are detected.

38. The method according to claim 36, wherein cells which have not been immobilized are removed, by washing the support plate, before the cell properties are detected.

39. A kit, comprising a support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules, to which the cells can bind by means of the cell surface molecules thereby being immobilized at the measuring points, and
raised sections which are spatially separated from each other and on which at least one measuring point is in each case provided.

40. A kit according to claim 39, wherein the measuring points have a diameter of between about 200 μm and about 4000 μm.

41. A kit, comprising a support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules to which the cells can bind by means of the cell surface molecules thereby being immobilized at the measuring points, and
raised sections which are spatially separated from each other and on which at least one measuring point is in each case provided,
wherein the trapping molecules comprise ligands for cell surface receptors, and the measuring points have a diameter of between about 200 μm and about 4000 μm, and an edge spacing of between about 300 μm and about 700 μm.

42. A kit, comprising a support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules to which the cells can bind by means of the cell surface molecules thereby being immobilized at the measuring points, and
raised sections which are spatially separated from each other and on which at least one measuring point is in each case provided, wherein each raised section is designed in the manner of a peg and is essentially planar at its facing surface, and
wherein the trapping molecules comprise ligands for cell surface receptors, and the measuring points have a diameter of between about 200 μm and about 4000 μm, and each peg has an area of 1-5 mm² at its facing surface.

43. The kit according to claim 39, further comprising a test substance.

44. The kit according to claim 40, further comprising a test substance.

45. The kit according to claim 41, further comprising a test substance.

46. The kit according to claim 42, further comprising a test substance.

47. A support plate for carrying out functional tests on biological cells, comprising an array of measuring points at which the cells can interact and bind, wherein the measuring points are separated from each other by regions of the support plate at which the cells cannot be immobilized, and wherein the measuring points are covered with trapping molecules, to which the cells can bind by means of the cell surface molecules thereby being immobilized at the measuring points, and, wherein a test substance is immobilized at the measuring points.

48. A kit, comprising:
a support plate for carrying out functional tests on biological cells, comprising
an array of measuring points at which the cells can interact and bind, wherein the measuring points are separated from each other by regions of the support plate at which the cells cannot be immobilized, and wherein the measuring points are covered with trapping molecules, to which the cells can bind by means of the cell surface molecules thereby being immobilized at the measuring points, and
raised sections which are spatially separated from each other and on which at least one measuring point is in each case provided, wherein each raised section is designed in the manner of a peg and is essentially planar at its facing surface, and
a metering plate, said plate comprising drilled holes which correspond to the pegs of said measuring plate such that, when the metering plate is mounted on the support plate, the pegs seal off, by means of their facing surfaces, the drilled holes towards the bottom of the support plate, thereby forming wells.

49. The method according to claim 31, wherein prior to applying the cells to the support plate, wells are formed over the pegs by mounting on a metering plate, said metering plate comprising drilled holes which correspond to the pegs of the support plate such that, when the metering plate is mounted on, the pegs seal off, by means of their facing surfaces, the drilled holes towards the bottom of the support plate, thereby forming wells.
